(19) United States
(12) Patent Application Publication
(10) Pub. No.: US 2008/0039828 A1
(43) Pub. Date: Feb. 14, 2008

(54) Laser Tissue Vaporization

(76) Inventors: Jose W. Jimenez, Apple Valley, MN (US); Gerald Mitchell, San Jose, CA (US); Christopher P. Olig, Minnetonka, MN (US); Scott E. Jahns, Hudson, WI (US)

Correspondence Address: AMS RESEARCH CORPORATION, 10700 BREN ROAD WEST, MINNETONKA, MN, 55343, US

(21) Appl. No.: 11/837,052

(22) Filed: Aug. 10, 2007

Related U.S. Application Data
(60) Provisional application No. 60/822,016, filed on Aug. 10, 2006.
(60) Provisional application No. 60/863,891, filed on Nov. 1, 2006.
(60) Provisional application No. 60/864,198, filed on Nov. 3, 2006.

(51) Int. Cl. A61B 18/20

(52) U.S. Cl. 606/13

(57) ABSTRACT

A system and related methods of use for selectively vaporizing targeted tissue. The system includes a laser capable of emitting a particular wavelength of laser light, a biocompatible colorant selected to absorb the particular wavelength and an injection device for tinting targeted tissue with the biocompatible colorant. The use of a laser tuned to selectively vaporize tinted, targeted tissue is especially suited to treatment of a wide range of medical conditions including effecting minimally invasive treatment of male reproductive organs and/or female reproductive organs to effect contraception, sterilization or fibroid removal.

### PRIORITY CLAIM

The present application claims priority to U.S. Provisional Application Ser. Nos. 60/822,016 filed Aug. 10, 2006; 60/863,891 filed Nov. 1, 2006 and 60/864,198 filed Nov. 3, 2006, each of which are hereby incorporated by reference in their entirety.

### FIELD OF THE DISCLOSURE

This invention relates to the field of laser treatment of soft tissue. More specifically, the invention is directed to the use of a laser to vaporize tissue to treat a variety of conditions, particularly in the minimally invasive treatment of male reproductive organs and/or female reproductive organs to effect contraception or sterilization.

### BACKGROUND OF THE INVENTION

Conventional strategies for treating conditions such as incontinence, prolapse, fibroids, erectile dysfunction, as well as contraception and sterilization, can involve surgery and/or drug therapies. When surgical methods are used to treat the above-noted conditions, often requiring the removal of tissue, the procedure can involve hospital time, be painful, can be expensive and require relatively long recovery time. Drug therapies may not be as effective in treating the condition as surgery can be, or the drug therapy can have undesirable, and potentially debilitating, side-effects. Contraceptive and sterilization strategies are good examples where a number of different surgical and drug strategies and techniques have been developed to achieve the desired result.

Conventional contraceptive strategies generally fall within three categories: physical barriers, drugs and surgery. While each have certain advantages, they also suffer from various drawbacks. Barriers such as condoms, sponges, and diaphragms are subject to failure due to breakage, displacement and misuse. Drug strategies, such as birth control pills and NORPLANT™, which rely on artificially controlling hormone levels, suffer from known and unknown side-effects from prolonged use. Finally, surgical procedures, such as tubal ligation and vasectomy, involve the costs and attendant risks of surgery, and are frequently not reversible.

In response to the aforementioned difficulties and inefficiencies of conventional contraceptive strategies, a number of implantable and permanent sterilization products have been developed to physically block the passage of reproductive cells between the ovary and the uterus. Representative products include those available under the trademarks OVION ECLIPSE® from American Medical System of Minnetonka, Minn. and ESSURE® permanent birth control available from Conceptus, Inc. of San Carlos, Calif. Generally, these implantable permanent sterilization products are positioned within the fallopian tube so as to promote tissue ingrowth, and over time, they physically occlude the fallopian tube.

Implantable, non-permanent, sterilization products are also available, where the implanted product can be modified to allow passage of sperm or ovum. Generally, occluding a reproductive tract or lumen to prevent the passage of reproductive cells through the lumen is accomplished by positioning an occluding member in the lumen. The occluding can be positioned in the fallopian tubes of the female reproductive tract or in the lumen of the vas deferens of the male reproductive tract. As described in U.S. Pat. Nos. 6,432,116; 6,096,052; and 7,073,504 to Callister et al., which are hereby incorporated by reference, an expandable occluding member can be placed within the body lumen of the fallopian tube or the vas deferens, and the expanded occluding member can be secured to the wall of the body lumen. The occluding member occludes the reproductive body lumen sufficiently to prevent the passage of reproductive cells therethrough.

The contraceptive method described above, using an occluding member, can be reversed. The occluding member can be reopened by, for example, collapsing the occluding member about a plug or mandrel and, when the process is to be reversed, the plug can be removed by laparoscopic or other instruments to reopen the passageway. A balloon dilatation catheter can be used to further expand the opening once the plug is removed.

U.S. Pat. No. 6,712,810 to Harrington et al., incorporated herein by reference, describes another method and device for occlusion of the fallopian tubes, wherein the lining of the utero-tubal junction is thermally damaged, followed by the placement of a reticulated foam plug. In one example, vascularized tissue grows into the plug and prevents or discourages the formation of scar tissue around the plug. If a relatively small foam pore size is used, it encourages formation of a vascularized capsule around the plug, which limits foreign body response so that the capsule does not constrict around the plug.

When permanent contraception, that is, sterilization is the desired outcome, sterilization of humans and animals is generally accomplished by using tubal ligation or tubal occlusion techniques for females or lumen ligation or lumen occlusion techniques for males in the form of vasectomies or clips to close off the lumen where the sperm or ovum travels. The procedures can be time-consuming, invasive, painful, and can include significant recovery time.

Hence, there remains a need for a method of performing tissue removal, in particular, sterilizations in a human or animal that is prompt, minimally invasive, with highly effective post treatment, to avoid subsequent follow-up and observation. There also remains a need for a safe, effective method of contraception, particularly a minimally invasive, non-surgical, method which is reversible.

### SUMMARY OF THE INVENTION

The present disclosure is directed to the use of a laser to vaporize tissue to treat a variety of conditions, including, but not limited to incontinence, prolapse, fibroids, and erectile dysfunction, as well as for contraception and sterilization. In various representative embodiments, a KTP (potassium-titanyl-phosphate) laser can be used to, for example, improve blood flow in the groin area by removing tissue that is obstructing blood flow hence causing erectile dysfunction; to remove the prostate followed by use of an anastomosis catheter; to eliminate small/medium uterine fibroids or hemorrhoids; to necrose tissue through a vaginal incision (or perineal for males) to cause scarring in the abdominal area to simulate what mesh does to help cure incontinence or prolapse; to remove the outer layer of the uterus to eliminate menorrhagia; to vaporize other tissue masses (e.g. cysts) in the gastrointestinal tract or other parts of the body; to conduct internal tubal ligations or tissue scarring to naturally create reversible occlusions in the fallopian tubes or to open the opening to the fallopian tube; and to pinpoint and sever the vas deferens to perform minimally invasive male sterilization.

In one aspect of the disclosure, a method of treating tissue comprises providing a solid-state laser and delivering the laser light to targeted tissue, wherein the targeted tissue has been subjected to a biocompatible colorant. Various solid state lasers can be used for this purpose, including a Q-switched arc lamp-pumped or a flash lamp-pumped laser using a frequency doubling crystal such as potassium-titanyl-phosphate (KTP). The pulse duration of the laser light is in the range of 0.1 to 500 milliseconds, and the wavelength of the laser light is preferably between 200 and 1100 nanometers. The laser light can be delivered to the targeted biocompatible colorant-containing tissue through an optical fiber or other delivery system. In particular, the KTP laser produces 532 nm light and, at high powers, can induce a superficial char layer in the biocompatible colorant-containing tissue that assists in the tissue strongly absorbing the laser light. Non-linear crystals such as lithium triborate (LBO) and beta barium borate (BBO) also produce 532 nm light.

In another aspect of the disclosure, male sterilization can be accomplished by severing the vas deferens using laser light tuned to a biocompatible colorant, the biocompatible colorant including but not limited to a dye, tint or chromophore, that is injected in the vicinity of the vas deferens. The laser is tuned/targeted to the biocompatible colorant and only vaporizes the tissue that is tinted with the biocompatible colorant. The vas deferens can be palpated and a needle can be inserted through the scrotum and, as the needle is being pulled back from the vicinity of the vas deferens, the biocompatible colorant can be released in a small pocket of tissue surrounding the vas deferens. The biocompatible colorant remains in the track left by the needle during the time of treatment. A laser fiber can be inserted in the vicinity of the biocompatible colorant and only the tissue tinted by the biocompatible colorant is vaporized. Laser light, such as green light (from a KTP laser) at 532 nm, holmium at 1064 nm, thulium, or other appropriate wavelength laser light, can be used. The vas deferens can be severed or damaged sufficiently to cause the walls of the vas deferens to collapse and become welded together to close off the vas deferens lumen. In some embodiments, the procedure can be performed at several points along the vas deferens lumen to ensure that the sterilization is complete and secure.

In another aspect, it is not necessary to insert a needle through the scrotum to deliver the biocompatible colorant. Alternatively, the biocompatible colorant can be injected in the vicinity of the vas deferens by positioning the needle through the use of imaging equipment and related techniques. Here, too, the biocompatible colorant can be released in a small pocket of tissue surrounding the vas deferens.

In yet another aspect of the present disclosure, it is not necessary to insert a laser fiber in the vicinity of the biocompatible colorant to activate the biocompatible colorant to vaporize the tissue. Alternatively, the laser light can be focused from the exterior of the body, so that the laser light hits the tinted biocompatible colorant containing tissue area. The biocompatible colorant absorbs the laser light and, consequently, the biocompatible colorant-containing tissue is vaporized.

The process of male sterilization through laser targeting of tinted, biocompatible colorant containing tissue can also be reversible. For example, laparoscopic or other similar minimally invasive instruments can be used to reopen the passageway, and a balloon dilatation catheter can be used to expand the opening. Alternatively, if the vas deferens lumen has been severed, anastomosis devices and methods can be used to reconnect the vas deferens lumen.

In the various previously described embodiments as well as those that follow, laser targeting of tinted, biocompatible colorant containing tissue can make use of the Greenlight system from the Laserscope division of American Medical Systems of Minnetonka, Minn., as well as other laser systems of appropriate wavelength. Generally, these laser systems can offer treatments in about the same or less time as current forms of male sterilization, and can be less invasive than current forms of male sterilization. The lumen of the vas deferens does not need to be accessed, as when a tubal ligation method or an occlusion member insertion method is used. Further, the biocompatible colorant does not need to be precisely placed and can be placed in multiple locations in the vicinity of the vas deferens.

In another aspect of the present disclosure, sterilization of a female can be accomplished by severing the fallopian tubes using laser light tuned to a biocompatible colorant that is injected in the vicinity of each of the fallopian tubes. The laser can be tuned to the biocompatible colorant and only vaporizes the tissue that is tinted with the biocompatible colorant. The biocompatible colorant can be injected through the cervix and up into the fallopian tubes in a pocket of tissue surrounding the fallopian tube and/or directly into the fallopian tube. A laser fiber can be inserted in the vicinity of the biocompatible colorant in the fallopian tube or in the vicinity of the biocompatible colorant-containing tissue around the segment of fallopian tube. The laser fiber is actuated and the laser light focuses on the tinted, biocompatible colorant-containing tissue and only this tissue tinted with the biocompatible colorant is vaporized. Thus, the fallopian tube is cut, similar to a tubal ligation. The fallopian tubes can be severed or damaged sufficiently to cause the walls of the fallopian tubes to collapse together. Then, the walls of the fallopian tube become welded together to close off the fallopian tube lumen. The biocompatible colorant can be injected at a number of locations along the fallopian tube, thus the laser fiber can be repositioned to sequentially vaporize the tissue at several locations along the fallopian tube, collapsing the fallopian tube lumen at multiple locations to ensure that sterilization is complete and secure. In some embodiments, the biocompatible colorant can be injected percutaneously, with the assistance of suitable medical imaging equipment and technology, to deliver the biocompatible colorant to the appropriate tissue in the vicinity of the fallopian tubes. Here, too, the biocompatible colorant is released into a small pocket of tissue in the vicinity of a segment of the fallopian tube, or in the fallopian tube.

In another aspect, it is not necessary to insert a laser fiber in the vicinity of the biocompatible colorant to activate the biocompatible colorant to vaporize the tissue but instead, the laser light can be focused from the exterior of the body, that is, percutaneously, so that the laser fiber does not need to be inserted through the cervix to be able to pinpoint the tinted, biocompatible colorant-containing tissue. The biocompatible colorant absorbs the laser light and, consequently, the tinted, biocompatible colorant-containing tissue is vaporized.

In yet another aspect, the laser vaporization of tinted, biocompatible colorant containing tissue to accomplish female sterilization can also be reversible. For example, laparoscopic or other similar minimally invasive instruments can be used to reopen the passageway, and a balloon dilatation catheter can be used to expand the opening.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE DRAWINGS

These as well as other objects and advantages of this invention, will be more completely understood and appreciated by referring to the following more detailed description of the presently preferred exemplary embodiments of the invention in conjunction with the accompanying drawings of which:

FIG. 1 is a block diagram illustration of a representative KTP laser.

FIG. 2 is an illustration of the male reproductive organs.

FIG. 3 is an illustration of the male reproductive organs showing use of a laser fiber.

FIG. 4 is an illustration of the female reproductive organs, showing application of biocompatible colorant percutaneously.

FIG. 5 is an illustration of the female reproductive organs, showing application of biocompatible colorant to the fallopian tube.

FIG. 6 is an illustration of the female reproductive organs, showing use of a laser fiber.

FIG. 7 is an illustration of a balloon catheter in a lumen.

FIG. 8 is an illustration of the male reproductive organs showing use of an anastomosis device and a balloon catheter.

FIG. 9 is an illustration of the male reproductive organs showing use of a balloon catheter.

FIG. 10 is an illustration of the female reproductive organs showing use of a balloon catheter.

FIG. 11 is an illustration of the female reproductive organs showing use of an anastomosis device.

FIG. 12 is an illustration of the female reproductive organs showing use of a laser fiber to remove fibroids.

FIG. 13 is an illustration of the female reproductive organs showing use of a catheter-positioned injector and a laser fiber to remove fibroids.

### DETAILED DESCRIPTION OF EMBODIMENTS OF THE INVENTION

As will be described in detail throughout the present specification, the utilization of laser light on tinted, biocompatible colorant-containing tissue to vaporize or ablate the tissue has many uses, such as, for example, improving blood flow in the groin area by removing tissue that is obstructing blood flow hence causing erectile dysfunction; removing the prostate followed by use of an anastomosis catheter; eliminating small/medium uterine fibroids or hemorrhoids; necrosing tissue through a vaginal incision (or perineal for males) to cause scarring in the abdominal area to simulate what mesh does to help cure incontinence or prolapse; removing the outer layer of the uterus to eliminate menorrhagia; vaporizing other tissue masses (e.g. cysts) in the gastrointestinal tract or other parts of the body; conducting internal tubal ligations or tissue scarring to naturally create reversible occlusions in the fallopian tubes or to open the opening to the fallopian tube; and pinpointing and severing the vas deferens to perform minimally invasive male sterilization. Contraception/sterilization methods are described below, as examples demonstrating the use of the invention; however the examples are not intended to be limiting.

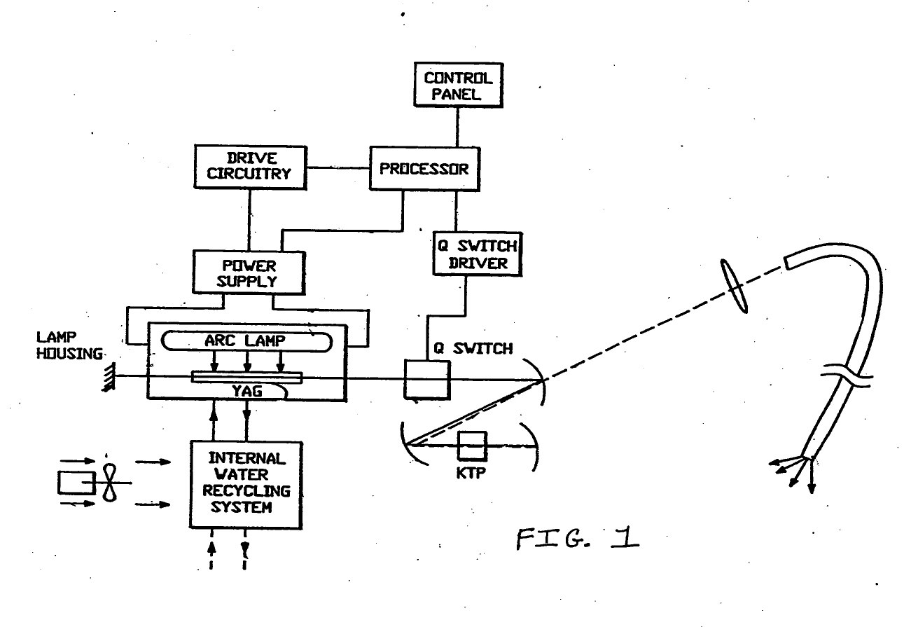

FIG. 1 shows a block diagram of an Nd:YAG surgical laser system utilizing a KTP crystal. However, it is noted that other appropriate lasers can be used in the methods and procedures described herein and the disclosed methods are not limited to the use of a KTP laser. YAG lasers use a yttrium-aluminum-garnet crystal rod as the lasing medium, with neodymium atoms dispersed in the YAG rod. The KTP crystal (potassium-titanyl-phosphate) is mounted in the optical path inside the laser resonator in order to extract harmonics of the frequency of the resonating beam or other frequencies of light by summing and/or subtracting various light beams. The laser is designed for resonating at a first frequency (e.g. 1064 nm) and a second frequency derived from the 1064 nm generated in the KTP crystal. The surgical laser uses the KTP crystal to extract a second harmonic 532 nm green output from a 1064 nm Nd:YAG laser source.

The output beam of the Nd:YAG laser, with KTP crystal, is delivered to a patient's tissue through an optical fiber or other delivery system. The main advantage of the 532 nm wavelength is that it is strongly absorbed by the hemoglobin in blood and hence useful for cutting, vaporizing and coagulating vascular tissue. A frequency doubled Nd:YAG laser suitable for such uses is described by P. E. Perkins and T. S. Fahlen in JOSA, Vol. 4, pp. 1066-1071 (1987), and advanced designs are described in U.S. Pat. No. 4,907,235 to Kuizenga, U.S. Pat. No. 5,151,909 to Davenpot et al.; U.S. Pat. No. 5,243,615 to Ortiz et al., U.S. Pat. No. 6,554,824 to Davenport et al.; U.S. Pat. No. 6,554,825 to Murray et al.; and U.S. Pat. No. 6,986,764 to Davenport et al., all of which are hereby incorporated by reference.

The duration of the laser pulse is variable and the laser can operate in continuous wave (CW) or pulsed mode. Continuous wave lasers emit a steady beam for as long as the laser medium is excited. Healing can be delayed and scarring can be increased if the steady laser beam is held on tissue longer that the thermal relaxation time, whereby excessive heat can be conducted into normal tissue. Pulsed lasers emit light in individual pulses. These pulses can be long pulsed (thousandths of a second) or short pulsed (millionth of a second). Q-Switching allows the laser to store energy between pulses. Hence, Q-Switching enables very high power output.

Generally, in the instance of an Nd:YAG laser with a KTP crystal (potassium-titanyl-phosphate) the wavelength of the laser, which is 1064 nm or 1320 nm, is halved to 532 nm. A green light can be used in continuous wave mode to cut tissue. In pulsed mode, the laser can be used for vascular lesions such as facial and leg veins. Other appropriate lasers can be used in the methods described herein.

The biocompatible colorant that is to be used with the selected laser must be complementary to the laser light produced, capable of absorbing the selected wavelength of laser light. For example, when a KTP laser producing green laser light is used in the methods described herein, a red biocompatible colorant for the target tissue is used. Examples of red biocompatible colorants that can be used with a KTP laser include Rhodamine 6G, carmine, Allura Red AC, Alizarin Red S and others. Biocompatible colorants can be obtained from chemical suppliers such Sigma-Aldrich and PolySciences, Inc.

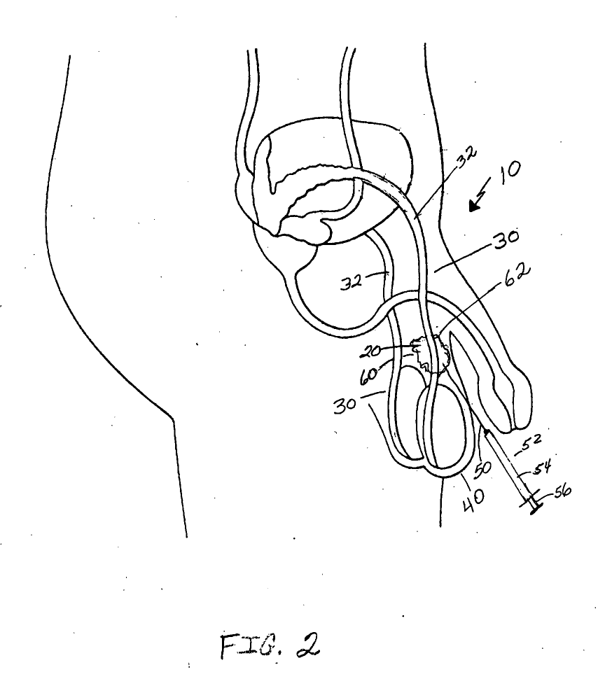

FIG. 2 illustrates the male reproductive organs 10, and the positioning of the biocompatible colorant 20 in the vicinity of the vas deferens 30. In one embodiment, the vas deferens 30 is palpated, that is, the vas deferens 30 is examined, and an injector, such as a needle 50, is inserted through the scrotum 40 such that the end of the needle 50 is in the vicinity of the vas deferens 30. A catheter can be utilized to position the injector/needle 50 in the vicinity of the vas deferens. The needle 50 is then removed from the biocompatible colorant tinted tissue 66 near the vas deferens 30. The biocompatible colorant 20 will generally remain in the small tissue pocket 62 during the treatment period. Alternatively, the biocompatible colorant 20 is injected in the vicinity of the vas deferens 30 percutaneously, with the use of imaging equipment. The desired location in the vicinity of the vas deferens 30 is identified, the needle 50 is entered into the targeted tissue 60 and the biocompatible colorant 20 is then deposited in the biocompatible colorant tinted tissue 66. The needle 50 is part of a syringe 52, the syringe consisting of a hollow barrel 54 fitted with a plunger 56 and a hollow needle 50. The hollow barrel 54 of the syringe 52 contains the biocompatible colorant that is to be injected into tissue 60 in the vicinity of the vas deferens 30. The needle 50 is positioned in the vicinity of the vas deferens 30 and the plunger 56 is depressed, thereby releasing the biocompatible colorant 20 in a small tissue pocket 62 surrounding the vas deferens 30. Preferably, the biocompatible colorant 20 is injected as the needle 50 is being withdrawn, such that the biocompatible colorant 20 is distributed over the track of the needle 50. The needle 50 is withdrawn and the method continues as described below.

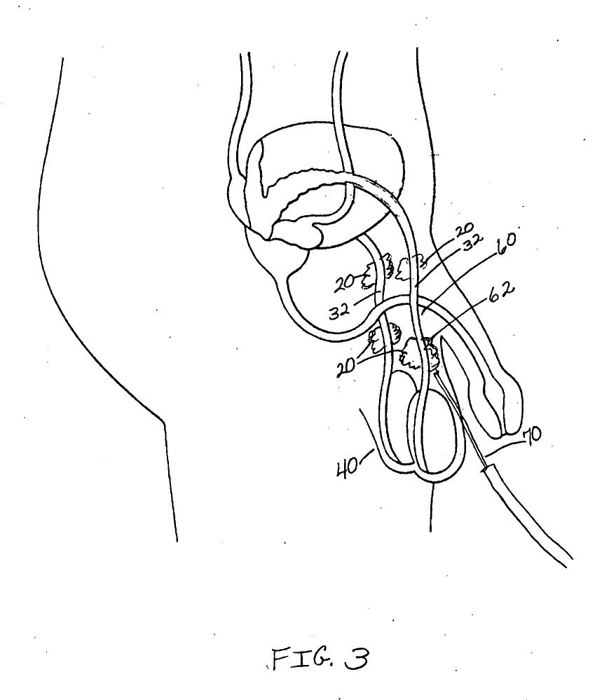

FIG. 3 illustrates a laser fiber 70 inserted through the scrotum 40 and positioned in the vicinity of the biocompatible colorant tinted tissue 66, in the vicinity of the vas deferens 30. A catheter can be used to position the laser fiber 70. The laser fiber 70 is activated and laser light tuned to the biocompatible colorant 20 focuses on the biocompatible colorant tinted tissue 66. The biocompatible colorant tinted tissue 66 absorbs the laser light and the biocompatible colorant tinted tissue 66 is ablated or vaporized. The surrounding tissue 60 that does not contain the biocompatible colorant 20 is not damaged. The vaporized biocompatible colorant tinted tissue 66 at the vas deferens 30 causes the vas deferens 30 to be severed or damaged sufficiently to cause the walls of the vas deferens lumen 32 to collapse together. When the walls of the vas deferens lumen 32 become welded together, the lumen 32 is effectively blocked to prevent the passage of sperm. Hence, sterilization of the male has been accomplished.

FIG. 3 illustrates another embodiment, wherein multiple injections of biocompatible colorant 20 into small tissue pockets 62 are effected at various positions along the vas deferens 30. The laser fiber 70 is inserted through the scrotum 40 and positioned in the vicinity of the first area of biocompatible colorant tinted tissue 66. The laser fiber 70 is activated and the laser light centers on the biocompatible colorant tinted tissue 66. The biocompatible colorant tinted tissue 66 is vaporized and the walls of the vas deferens lumen 32 are collapsed. The laser fiber 70 is repositioned to the next area of biocompatible colorant tinted tissue 66, and the laser fiber 70 is activated. As before, the laser light focuses on the biocompatible colorant tinted tissue 66 and vaporizes the biocompatible colorant tinted tissue 66. This process is continued until all of the biocompatible colorant tinted tissue 66 is subjected to the laser light and the biocompatible colorant tinted tissue 66 is vaporized. In each vaporization step, the walls of the vas deferens 30 lumen 32 collapse proximate the biocompatible colorant tinted tissue 66. Thus, tissue vaporization can be accomplished sequentially at several points along the vas deferens lumen 32, collapsing the walls of the lumen 32, and ensuring that sterilization is complete and secure.

In yet another embodiment, male sterilization is accomplished without having to insert the laser fiber through the scrotum 40 to position the laser fiber in the vicinity of the biocompatible colorant tinted tissue 66 near the vas deferens 30. Instead of accessing the biocompatible colorant tinted tissue 66 internally, the laser fiber 70 is positioned on the exterior of the scrotum 40 in the vicinity of the biocompatible colorant tinted tissue 66. The laser light from the laser fiber 70 focuses on the biocompatible colorant tinted tissue 66 and vaporizes the biocompatible colorant tinted tissue 66. The surrounding tissue 60, which has not been tinted or otherwise colorized with biocompatible colorant 20, between the laser light and the biocompatible colorant tinted tissue 66 is left largely unaffected. To more effectively and efficiently vaporize the biocompatible colorant tinted tissue 66, more than one laser fiber 70 can be used to triangulate the laser light to hit the biocompatible colorant tinted tissue 66 without having to insert the laser fiber 70 in the body.

A Greenlight laser system providing laser light at 532 nm is used for the sterilization procedure described above. However, other laser light systems can be utilized in the sterilization process. For example, a holmium laser providing 1064 nm wavelength laser light, or a thulium laser can be used in the sterilization process, so long as the laser light wavelength and the wavelength at which the biocompatible colorant is excited are compatible.

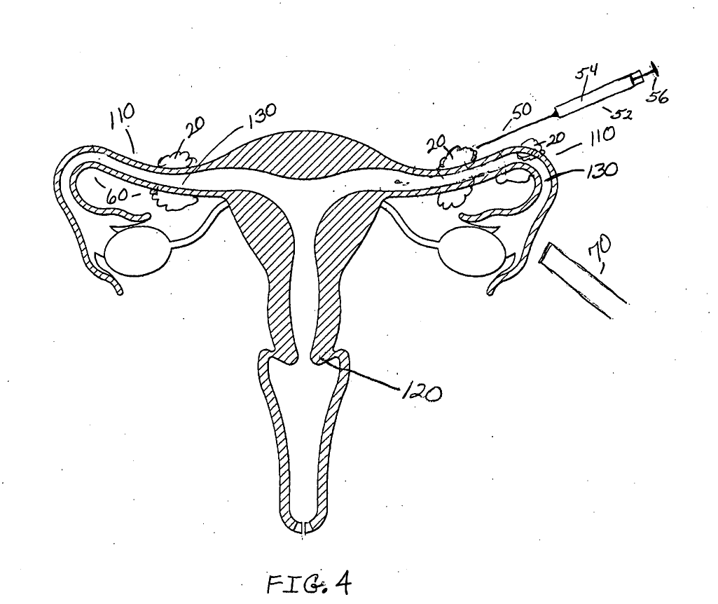

In another embodiment, the laser light system is used to accomplish female sterilization. FIG. 4 illustrates the reproductive organs 100 of a human female. To effect sterilization of the female, the fallopian tubes 110 must be blocked such that ovum cannot travel down the tube and/or sperm cannot travel up the fallopian tubes 110. The fallopian tubes 110, therefore, are severed by the use of laser light, such as by using the Greenlight laser system from Laserscope/AMS.

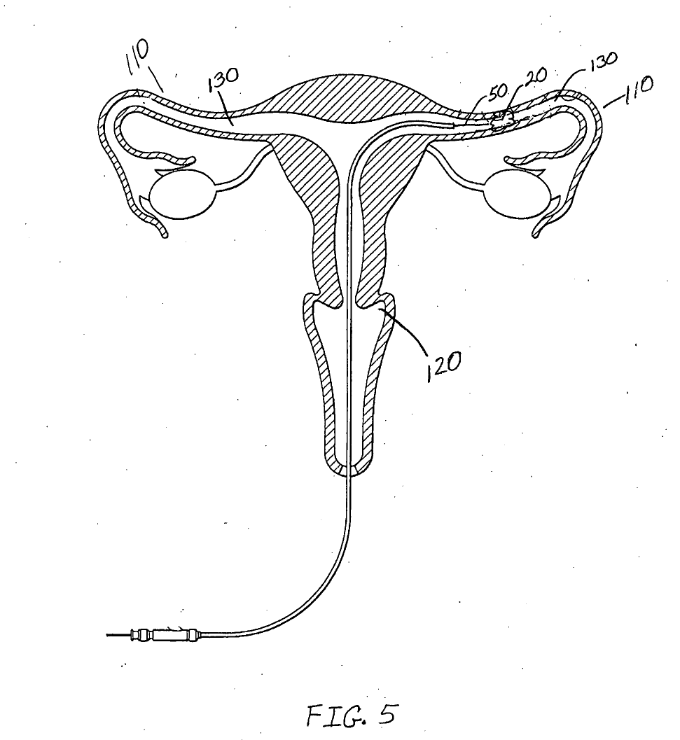

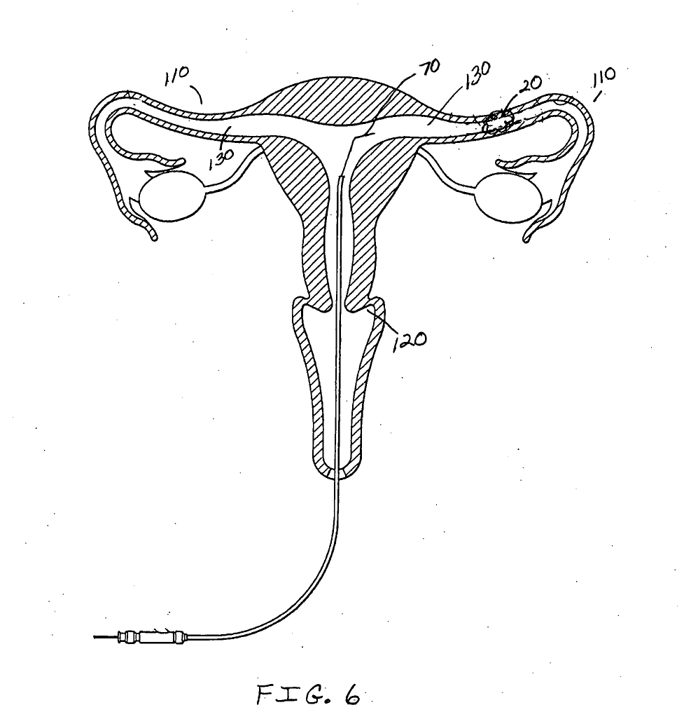

Referring to FIGS. 5 and 6, the fallopian tubes 110 are accessible by an injector such as a needle 50, as well as by a laser fiber 70, through the cervix 120. A catheter can be used to position the needle 50 and to position the laser fiber 70. A needle 50 is inserted through the cervix 120 such that the end of the needle 50 is either in the vicinity of the fallopian tubes 110 or within the fallopian tube 110. The needle 50 is positioned, for example, in the fallopian tubes 110, and the biocompatible colorant 20 is released in the fallopian tube lumen 130. The needle 50 and delivery system are then removed from the fallopian tubes 110.

Alternatively, as shown in FIG. 4, the targeted tissue 60 in the vicinity of the fallopian tube 110 can be accessed by the biocompatible colorant-containing needle percutaneously. Imaging equipment is used to guide the needle 50 to the desired target tissue 60 in the vicinity of the fallopian tube 110. The needle 50 is part of a syringe 52, the syringe consisting of a hollow barrel 54 fitted with a plunger 56 and a hollow needle 50. The hollow barrel 54 of the syringe 52 contains the biocompatible colorant 20 that is to be injected into tissue 60 in the vicinity of the fallopian tubes 110. The plunger 56 of the syringe 52 is depressed and the biocompatible colorant 20 is released into the small tissue pocket 62 in the vicinity of the fallopian tube 110. The biocompatible colorant 20 remains in the small tissue pocket 62, in the biocompatible colorant tinted tissue 66, during the treatment period.

In one embodiment, the biocompatible colorant 20 is injected in the vicinity of the fallopian tubes 110, into tissue 60 surrounding a particular segment of fallopian tube 110. Alternatively, in another embodiment, the biocompatible colorant 20 is injected into the fallopian tube 110. In yet another embodiment, the biocompatible colorant 20 is injected in both the tissue 60 surrounding a particular segment of fallopian tube 110 as well as in the fallopian tube 110.

Once the biocompatible colorant tinted tissue 66 in the vicinity of the fallopian tube 110 and/or in the fallopian tube 110 is ready for vaporization, a laser fiber 70 is inserted in the vicinity of the biocompatible colorant tinted tissue 66 around a segment of fallopian tube 110 or in the fallopian tube 110. The laser fiber 70 is actuated and the laser light focuses on the biocompatible colorant tinted tissue 66 and only this biocompatible colorant tinted tissue 661 is vaporized/ablated. Thus, the fallopian tube 110 is severed, similar to a tubal ligation. The fallopian tubes 110 are severed or damaged sufficiently to cause the walls of each fallopian tube 110 to collapse together. The walls of the collapsed fallopian tube 110 become welded together to close off the fallopian tube lumen 130.

In another embodiment, the biocompatible colorant 20 is injected in a number of locations along the length of the fallopian tube 110, thus the laser fiber 70 vaporizes the biocompatible colorant tinted tissue 66 at multiple positions along the fallopian tube 110 to ensure that the sterilization is complete and secure. The laser fiber 70 can be repositioned to sequentially vaporize the biocompatible colorant tinted tissue 66. The walls of the fallopian tube 110 collapse in a number of locations, thus effectively blocking the fallopian tube lumen 130.

In another embodiment, female sterilization is accomplished without having to insert the laser fiber 70 through the cervix 120 to position the laser fiber 70 in the vicinity of the biocompatible colorant tinted tissue 66 near the fallopian tube 110, or in the fallopian tube 110. Instead of accessing the biocompatible colorant tinted tissue 66 internally, the laser fiber 70 is positioned on the exterior of the body, percutaneously, in the vicinity of the biocompatible colorant tinted tissue 66. The laser light from the laser fiber 70 focuses on the biocompatible colorant tinted tissue 66 and vaporizes the tinted tissue 66. The tissue 60 between the laser light and the biocompatible colorant tinted tissue 66 is left largely unaffected. To more effectively and efficiently vaporize the biocompatible colorant tinted tissue 66, more than one laser fiber 70 can be used to triangulate the laser light to hit the biocompatible colorant tinted tissue 66 without having to insert the laser fiber 70 in the body.

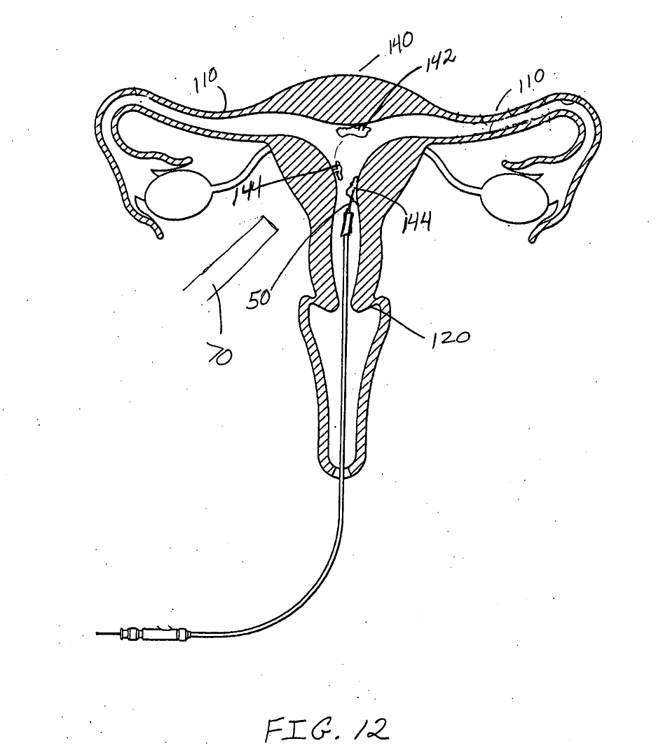

The embodiments presented have been focused on the sterilization of the human male and female. However, the techniques described herein can also be used on animals, as appropriate. Further, the techniques described above can be used for vaporizing or ablating other soft body tissue. For example, the techniques described above can be used for removing fibroids from the female reproductive system, in particular, from the uterus 140. FIG. 12 illustrates the presence of fibroids 142 in the uterus 140. Fibroids 142 are collagen-containing growths that can occur in various areas of the uterus. Fibroids 142 that develop in the outer portion of the uterus are called subserosal uterine fibroids; fibroids 142 that develop within the uterine wall are called intramural uterine fibroids; and fibroids 142 that develop just under the uterine cavity are called submucosal uterine fibroids.

Conventional treatment for uterine fibroids 142 typically involves the use of medications and/or surgery. Surgical procedures used to remove uterine fibroids 142 include hysterectomy, where the uterus is removed, and myomectomy. A hysterectomy is a fairly major type of surgery and recovery can be long and painful. A myomectomy, where fibroids are surgically removed from the uterus, can also result in time spent in the hospital.

FIG. 12 illustrates the use of laser light to remove uterine fibroids 142. The uterus 140 is accessible by an injector such as a needle 50, which can be positioned by a catheter, as well as by a laser fiber 70, through the cervix 120. A needle 50 is inserted through the cervix 120 such that the end of the needle 50 injects the biocompatible colorant 20 into the fibroid 142, resulting in a biocompatible colorant tinted fibroid 144. The needle 50 and delivery system are then removed from the uterus 140.

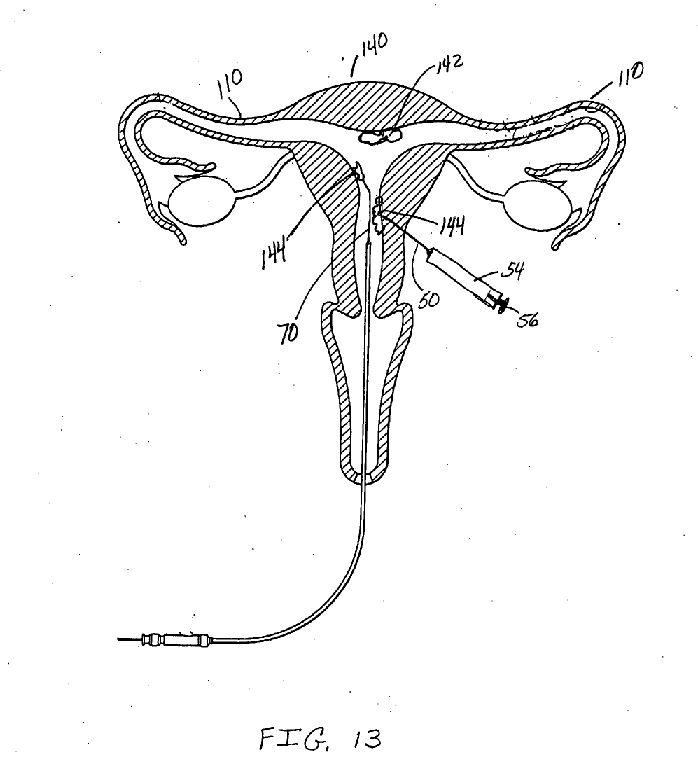

Alternatively, as shown in FIG. 13, the fibroid 142 can be accessed by the biocompatible colorant-containing needle 50 percutaneously. Appropriate imaging equipment is used to guide the needle 50 to the fibroid 142. The needle 50 is part of a syringe 52, the syringe consisting of a hollow barrel 54 fitted with a plunger 56 and a hollow needle 50. The hollow barrel 54 of the syringe 52 contains the biocompatible colorant 20 that is to be injected into the fibroid 142. The plunger 56 of the syringe 52 is depressed and the biocompatible colorant 20 is released into the fibroid 142. The biocompatible colorant tinted fibroid 144 is ready for removal through the use of laser light.

FIG. 13 shows the laser fiber 70 inserted through the cervix 120 to position the laser fiber 70 in the vicinity of the tinted fibroid 144. A catheter can be used to position the laser fiber 70 in the uterus 140. The laser is actuated and the laser light vaporizes the biocompatible colorant tinted fibroid 144, with little effect on surrounding tissue 60. Instead of accessing the biocompatible colorant tinted fibroid 144 through the cervix 120, the laser fiber 70 is positioned on the exterior of the body, percutaneously, in the vicinity of the biocompatible colorant tinted fibroid 144. The laser light from the laser fiber 70 focuses on the biocompatible colorant tinted fibroid 144 and vaporizes the biocompatible colorant tinted fibroid 144. The surrounding tissue 60 is left largely unaffected.

The selective tissue vaporization techniques described herein are also used as a means of providing contraception, in that the techniques allow for reversing the sterilization of the male and/or female. In the instance when sterilization is no longer desired and, instead, the individual desires the sterilization procedure to be reversed, the collapsed vas deferens lumen 32 in the male or the fallopian tube lumen 130 in the female can be expanded to once again function properly.

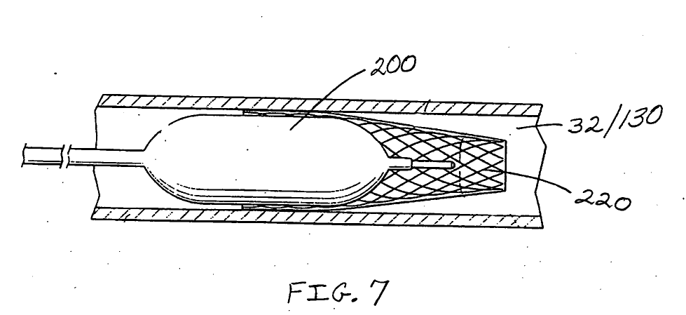

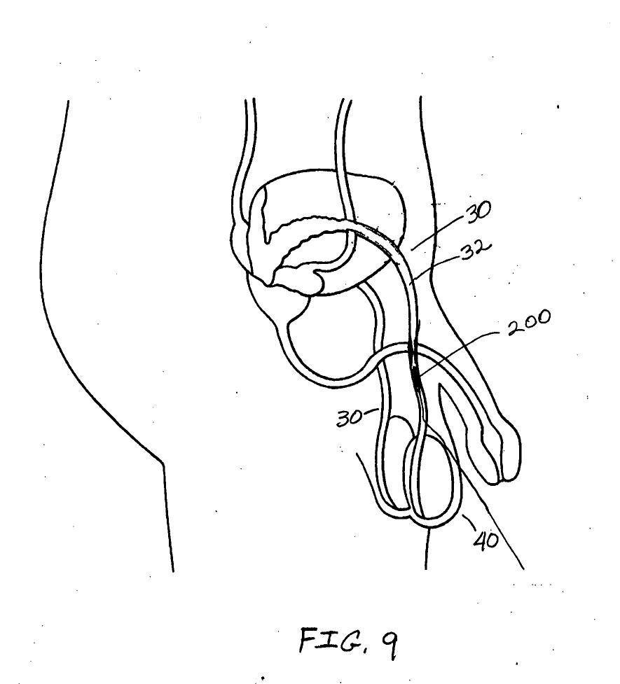

The sterilization of the male is reversed by the insertion of a balloon catheter 200, as shown in FIGS. 7 and 9, in the collapsed lumen 32 of the vas deferens 30. The balloon catheter 200 is inserted through the scrotum 40 and into the vas deferens 30. The balloon catheter 200 is advanced slowly within the vas deferens lumen 32 until the balloon is within the lumen that has been collapsed. The balloon catheter 200 is eased into the collapsed lumen and then the balloon on the catheter 200 is inflated, thus expanding the lumen 32 such that sperm can once again pass through the vas deferens lumen 32. If the collapsed vas deferens lumen 32 is not able to retain its expanded open configuration, as provided by the balloon catheter 200, a hollow tubular member 220 is inserted in the vas deferens lumen 32, such that the vas deferens lumen 32 retains its open configuration. Thus, the sterilization process is reversed.

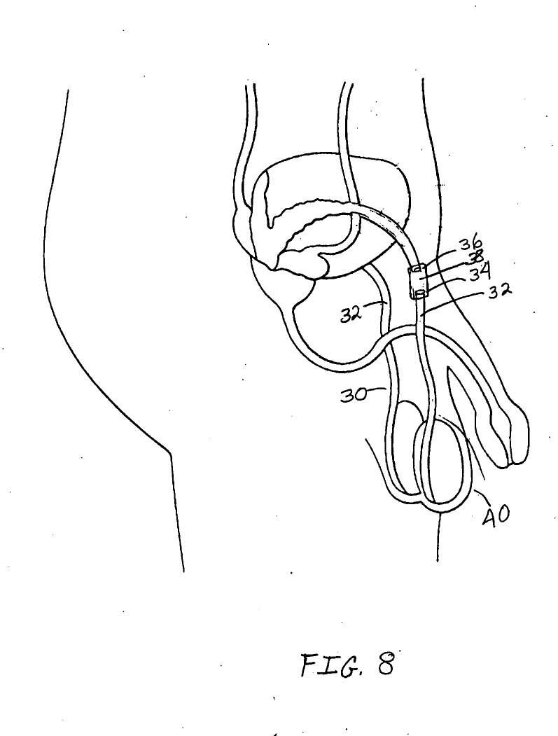

Alternatively, if the vas deferens lumen 32 has been severed, then an anastomosis device is used to repair the vas deferens lumen 32 as illustrated in FIG. 8. The anastomosis device has a tissue approximation structure 38 allowing for grasping an approximation of proximal vas deferens tube stumps 34 and distal vas deferens tube stumps 36 remaining from the sterilization procedure so as to restore a lumen 32 defined by the vas deferens 30 for subsequent passage of sperm. The anastomosis device includes a catheter body that is advanced through the scrotum 40 and the vas deferens lumen 32 and into the proximal stump 34. Further, the anastomosis device can include a flexible guidewire with a radioopaque tip viewable with a suitable medical imaging system such as, for example, a fluoroscopic imaging system. The guidewire is used to deliver the tissue approximation 38 structure to the proximal stump 34 such that a set of proximal approximating structure can be extended to grasp the proximal stump 34. The tissue approximation structure 38 is advanced into the distal stump 36 wherein a set of distal approximating structures can grasp the distal stump 36 and cause the proximal and distal stumps to be brought into contact so as to commence biological healing and restoration of the lumen defined by the vas deferens 30. If the vas deferens lumen 32 has been severed in a number of locations, the anastomosis device can be used in these multiple locations.

Further detail of anastomosis devices and methods are found in U.S. Published Patent Application Nos. 2004/0087995 A1; 2005/0070938A1, and 2005/0131431 A1 to Copa et al., and are all herein incorporated in their entireties by reference.

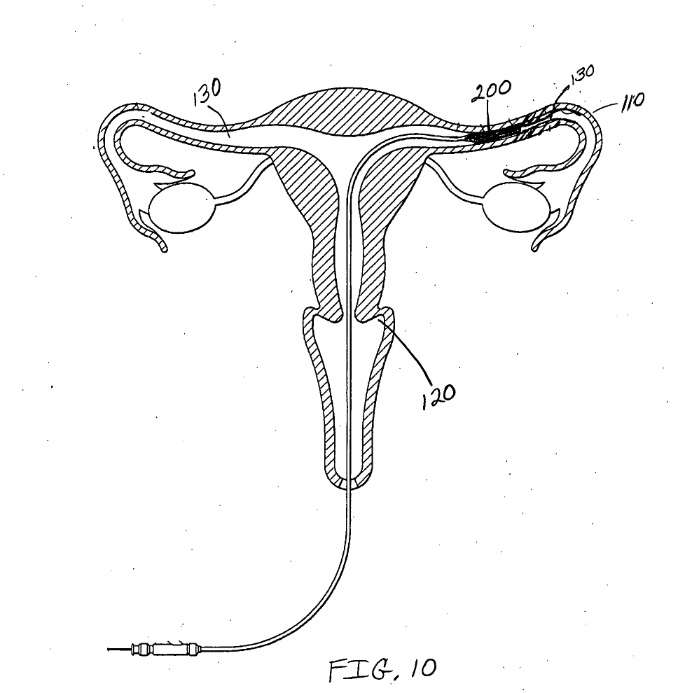

The sterilization of the female is reversed by the insertion of a balloon catheter 200 in the collapsed lumen 130 of the fallopian tube 110 as illustrated in FIG. 10. The balloon catheter 200 is inserted through the cervix 120 and into the fallopian tube 110. The balloon catheter 200 is advanced slowly within the fallopian tube lumen 130 until the balloon is within the lumen 130 that has been collapsed. The balloon catheter 200 is eased into the collapsed lumen and then the balloon on the catheter 200 is inflated, thus expanding the lumen 130 such that reproductive cells can once again pass through the fallopian tube lumen 130. If the collapsed fallopian tube lumen 130 is not able to retain its expanded open configuration, as provided by the balloon catheter 200, a hollow plug 220 is inserted in the fallopian tube lumen 130, such that the fallopian tube lumen 130 retains its open configuration. Thus, the sterilization process is reversed.

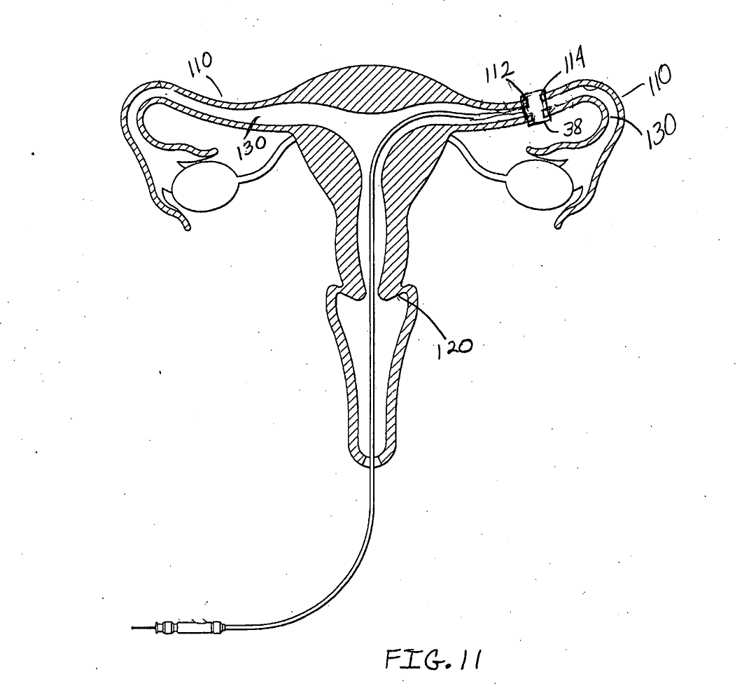

Alternatively, if the fallopian tube lumen 130 has been severed, then an anastomosis device is used to repair the fallopian tube lumen 130 as illustrated in FIG. 11. The anastomosis device has a tissue approximation structure 38 allowing for grasping an approximation of proximal fallopian tube stumps 112 and distal fallopian tube stumps 114 remaining from the sterilization procedure so as to restore a lumen defined by the fallopian tubes 110 for subsequent passage of reproductive cells. The anastomosis device includes a catheter body that is advanced through the cervix 120 and the fallopian tube lumen 130 and into the proximal stump 112. Further, the anastomosis device can include a flexible guidewire with a radioopaque tip viewable with a suitable medical imaging system such as, for example, a fluoroscopic imaging system. The guidewire is used to deliver the tissue approximation structure 38 to the proximal stump 112 such that a set of proximal approximating structure can be extended to grasp the proximal stump 112. The tissue approximation structure 38 is advanced into the distal stump 114 wherein a set of distal approximating structures can grasp the distal stump 114 and cause the proximal and distal stumps 112/114 to be brought into contact so as to commence biological healing and restoration of the lumen defined by the fallopian tube 110. If the fallopian tube lumen 130 has been severed in a number of locations, the anastomosis device can be used in these multiple locations. Hence, female sterilization using a laser based technique for selective tissue vaporization can be reversed using a balloon catheter or, alternatively, an anastomosis device.

Although specific examples have been illustrated and described herein, it will be appreciated by those of ordinary skill in the art that any arrangement calculated to achieve the same purpose could be substituted for the specific examples shown. This application is intended to cove adaptations or variations of the present subject matter. Therefore, it is intended that the invention be defined by the attached claims and their legal equivalents.

We claim:

1. A method for performing a sterilization procedure comprising:
providing a laser capable of emitting a selected wavelength of laser light;
selecting a biocompatible colorant for absorbing the selected wavelength of laser light;
depositing the biocompatible colorant proximate a portion of a reproductive lumen; and
actuating the laser such that laser light emitted from the laser is delivered to the biocompatible colorant to vaporize the portion of the reproductive lumen.

2. The method of claim 1, wherein depositing the biocompatible colorant comprises: injecting the biocompatible colorant into a tissue pocket adjacent to the portion of the reproductive lumen.

3. The method of claim 1, wherein depositing the biocompatible colorant comprises: injecting the biocompatible colorant into the portion of the reproductive lumen.

4. The method of claim 1, wherein the reproductive lumen comprises a female fallopian tube or a male vas deferens.

5. The method of claim 1, further comprising:
positioning the laser percutaneously such that the laser light is focused on the biocompatible colorant.

6. The method of claim 1, wherein the biocompatible colorant is deposited proximate a plurality of distinct portions of the reproductive lumen and wherein the laser is sequentially actuated to vaporize the distinct portions of the reproductive lumen.

7. The method of claim 1, wherein depositing the biocompatible colorant comprises percutaneously injecting the biocompatible colorant.

8. A method for selectively vaporizing tissue comprising:
providing a laser capable of emitting a selected wavelength of laser light;
selecting a biocompatible colorant for absorbing the selected wavelength of laser light;
depositing the biocompatible colorant proximate targeted tissue; and
actuating the laser such that laser light emitted from the laser is delivered to the biocompatible colorant to vaporize the targeted tissue.

9. The method of claim 8, wherein depositing the biocompatible colorant comprises:
injecting the biocompatible colorant into a tissue pocket adjacent the target tissue.

10. The method of claim 8, wherein depositing the biocompatible colorant comprises injecting the biocompatible colorant into the targeted tissue.

11. The method of claim 10, wherein the targeted tissue comprises a fibroid.

12. The method of claim 8, further comprising:
positioning the laser percutaneoulsy such that the laser light is focused on the biocompatible colorant.

13. The method of claim 8, wherein depositing the biocompatible colorant comprises percutaneously injecting the biocompatible colorant.

14. A system for selectively vaporizing tissue comprising:
a laser capable of emitting a selected wavelength of laser light;
a biocompatible colorant for absorbing the selected wavelength of laser light; and
an injector for depositing the biocompatible colorant within targeted tissue.

15. The system of claim 14 wherein the laser is selected from a group comprising: a KTP laser, a lithium triborate (LBO) laser, a beta barium borate (BBO), a holmium laser and a thulium laser.

16. The system of claim 14 wherein the wavelength of the emitted laser light ranges from about 200 nm to about 1100 nm.

17. The system of claim 14 further comprising a laser fiber capable of delivering a selected wavelength of laser light.

18. The system of claim 17 further comprising a catheter capable of minimally invasively positioning the laser fiber.

19. The system of claim 14 further comprising a catheter capable of minimally invasively positioning the injector.

20. The system of claim 14, wherein the targeted tissue is selected from the group consisting essentially of: a fallopian tube, a vas deferens and a fibroid.
